(19) United States
(12) Patent Application Publication
(10) Pub. No.: US 2012/0027745 A1
(43) Pub. Date: Feb. 2, 2012

(54) ALANINE-GLYOXYLATE AMINOTRANSFERASE THERAPEUTICS

(75) Inventors: Maria Sol Rodriguez Pena, Le Mont-sur-Lausanne (CH); Harald Petry, Amsterdam (NL); Jaap Twisk, Amsterdam (NL); Sander Jan Hendrik Van Deventer, Amsterdam (NL); Eduardo Carlos Salido Ruiz, Tenerife (ES); Armando Torres Ramirez, Tenerife (ES)

(73) Assignee: Amsterdam Molcular Therapeutics (AMT) IP B.V.

(21) Appl. No.: 13/146,869

(22) PCT Filed: Feb. 1, 2010
(86) PCT No.: PCT/NL2010/050044

§ 371 Date: Oct. 5, 2011

(51) Int. Cl. A61K 38/45; A61P 3/00; C12N 15/86; C12N 15/864; A61K 48/00; C12N 15/85

(52) U.S. Cl. 424/94.5; 514/44 R; 435/320.1

(57) ABSTRACT

The present invention relates to an alanine glyoxylate aminotransferase (AGXT) I340M therapeutic for use as a medicament or in a method of treatment, for example in the treatment of an AGXT-responsive condition. The AGXT I340M therapeutic is an AGXT I340M protein comprising an amino acid sequence, which, when optimally aligned with SEQ ID NO: 2, comprises a methionine at a position corresponding to position 340 in SEQ ID NO: 2, a nucleic acid molecule encoding such an AGXT I340M protein, or a virion of a viral gene therapy vector comprising such a nucleic acid molecule. The AGXT I340M therapeutic has a higher specific activity as compared to other AGXT alleles and may therefore be advantageously used in the treatment of primary hyperoxaluria type I.

### FIELD OF THE INVENTION

The present invention is in the fields of medicine and molecular biology. More specifically the invention relates to novel protein and nucleic acid therapeutics based on alanine-glyoxylate aminotransferase (AGXT), in particular a variant of AGXT, including therapeutics delivered by gene therapy, and methods for use of these therapeutics in treating and preventing conditions caused by absence or deficiency of alanine-glyoxylate aminotransferase, including primary hyperoxaluria type I.

### BACKGROUND OF THE INVENTION

Primary hyperoxaluria type I (PH1) (OMIM #259900) is a rare metabolic disorder, inherited in an autosomal recessive manner. PH1 is characterized by a deficiency of the hepatic enzyme alanine-glyoxylate aminotransferase (AGXT or AGT; EC 2.6.1.44), that is usually caused by a mutation in the gene encoding on 2q36-q37 (Genbank Acc. No. NM_{—}000030, version 14 Jun. 2008). An AGXT deficiency results in failure to detoxify glyoxylate, with overproduction of oxalate. AGXT converts glyoxylate to glycine, using alanine as the donor of the amino group, and the cofactor pyridoxal-phosphate. High levels of oxalate in PH1 patients are excreted by the kidneys, which undergo progressive deterioration as a result of calcium oxalate deposition. After kidney failure, oxalate levels raise to the point of systemic oxalosis, a life threatening condition. Currently, the most effective treatment for PH1 is pre-emptive liver transplantation or combined liver and kidney transplantation. However, this treatment has its own limitations including the scarce supply of suitable organs, significant morbidity and mortality, and chronic exposure to immunosuppressive drugs.

Thus, new treatments for PH1 are needed. It is an object of the present invention to provide to novel protein and nucleic acid therapeutics based on variants of alanine-glyoxylate aminotransferase (AGXT) and methods for use of these therapeutics in treating and preventing conditions caused by absence or deficiency of alanine-glyoxylate aminotransferase, including primary hyperoxaluria type I.

### SUMMARY OF THE INVENTION

The invention relates to an AGXT therapeutic, such as an AGXT I340M therapeutic, for use as a medicament or for use in a method of treatment. Such a therapeutic may be used in the treatment of an AGXT-responsive condition, whereby, preferably the AGXT-responsive condition is primary hyperoxaluria type I.

The AGXT I340M therapeutic preferably is selected from the group consisting of a) an AGXT I340M protein comprising an amino acid sequence having at least 90% sequence identity to SEQ ID NO: 2 when optimally aligned over their entire length using the GAP program, and wherein the AGXT I340M protein comprises a methionine at a position corresponding to position 340 in SEQ ID NO: 2; b) a nucleic acid molecule comprising an nucleotide sequence encoding an AGXT I340M protein as defined in a); and, c) a virion of a viral gene therapy vector comprising a nucleic acid molecule as defined in b).

An AGXT therapeutic based on the wild type may be defined in a similar fashion as to the AGXT I340M therapeutic described above, except that such a therapeutic would comprise isoleucine (or an amino acid other than methionine) at a position corresponding to position 340 in SEQ ID NO: 2 (i.e. may be based on the AGXT wild type sequence). Herein, that which is described in relation to an AGXT I340M therapeutic may be applied similarly to an AGXT therapeutic based on the wild type AGXT sequence.

An AGXT I340M therapeutic according to the invention may further comprise or encode amino acid substitutions selected from the group consisting of: a) a leucine at a position corresponding to position 11 in SEQ ID NO: 2; b) an arginine at a position corresponding to position 41 in SEQ ID NO: 2; c) an isoleucine at a position corresponding to position 152 in SEQ ID NO: 2; d) an arginine at a position corresponding to position 170 in SEQ ID NO: 2; e) a threonine at a position corresponding to position 244 in SEQ ID NO: 2; f) a threonine at a position corresponding to position 294 in SEQ ID NO: 2; g) an isoleucine at a position corresponding to position 326 in SEQ ID NO: 2; h) combinations of one or more of the substitutions amino acid b) to g); and, i) the combination of the substitutions a) and f).

The invention also relates to a nucleic acid construct comprising a nucleotide sequence coding for the AGXT I340M protein of the invention, wherein the nucleotide sequence is operably linked to a promoter for expression in human cells and wherein, optionally, the promoter is not a promoter from a human AGXT gene. Preferably, the promoter is a liver-specific promoter. The liver-specific promoter may be selected from the group consisting of an α1-anti-trypsin (AAT) promoter, a thyroid hormone-binding globulin promoter, an albumin promoter, a thyroxin-binding globulin (TBG) promoter, an Hepatic Control Region (HCR)-ApoCII hybrid promoter, an HCR-hAAT hybrid promoter, an AAT promoter combined with the mouse albumin gene enhancer (Ealb) element, an apolipoprotein E promoter and a promoter that has the sequence of SEQ ID NO: 3. The nucleic acid construct according to this aspect preferably is a viral gene therapy vector, more preferably a parvoviral vector.

The invention further pertains to a parvoviral virion comprising a nucleic acid construct encoding the AGXT I340M therapeutic.

Also, the invention relates to a pharmaceutical composition comprising an AGXT therapeutic, nucleic acid construct or parvoviral virion of the invention and a pharmaceutically acceptable carrier.

The invention also provides a method for treating an AGXT-responsive condition wherein the method comprises the step of administering an effective amount of an AGXT I340M therapeutic, a nucleic acid construct, a parvoviral virion or a pharmaceutical composition of the invention to a subject having a said AGXT-responsive condition, for example, primary hyperoxaluria type I.

In addition, the invention provides, use of an AGXT I340M therapeutic, a nucleic acid construct, a parvoviral virion or a pharmaceutical composition of the invention in the manufacture of a medicament for use in the treatment of an AGXT-responsive condition, for example, primary hyperoxaluria type I.

### DESCRIPTION OF THE FIGURES

FIG. 1 shows a schematic of the rAAV vectors used. rAAV vectors were constructed by inserting the human AGXT cDNA into a pro-AAV2 vector plasmid, under the control of a hybrid EalbAAT liver-specific promoter. Downstream of the AGXT cDNA (or GFP) a bGH polyadenylation signal was included. Some constructs included a WPRE post-transcriptional regulatory element. The expression cassette was flanked by inverted terminal repeats from AAV2

FIG. 2 shows urinary excretion of Oxalate in male AGXT^{-/-} mice treated with ethylene glycol (EG) after the administration of 5×10¹² gc/kg of therapeutic vector AAV8.Ealb-hAAT-AGXT-WPRE or control vector AAV8.Ealb-hAAT-GFP-WPRE.

FIG. 3a shows Western blot analysis of AGXT expression within livers of AGXT−/− mice (of both genders) 8 weeks after intravenous injection of 5×10e12 vector genomes per kg AAV8 or AAV5 vector (the latter also administered at the ten-fold lower dose 5×10e11vg/kg).

FIGS. 3b and 3c (the insert) show expression of AGT in liver samples from male AGXT^{-/-} mice injected with the AAV8.Ealb-hAAT-AGXT-WPRE or control vector AAV8.Ealb-hAAT-GFP-WPRE (100× magnification) respectively.

FIG. 3d shows results obtained by immunohistochemical staining of frozen liver sections, with percentages of hepatocytes transduced around 90% for AAV8 and the highest dose of AAV5, 60% for 5×10¹² AAV5 particles/kg, and down to 10% (and less than 1% in females) for 5×10¹¹ AAV5 particles/kg.

FIG. 4 shows urinary excretion of Oxalate in male AGXT^{-/-} mice treated with ethylene glycol (EG) after the administration of either 1×10¹⁰, 1×10¹¹ and 3×10¹¹ gc/kg of therapeutic vector AAV5.Ealb-hAAT-AGXT-WPRE or control vector AAV5.Ealb-hAAT-GFP-WPRE (at the day of injection and 27 days later). A marked reduction in oxaluria is observed in mice treated with all three doses tested, although higher doses showed the most statistically significant reduction, achieving oxalate excretion levels similar to wild type mice. As expected, AgxtKO mice injected with GFP-AAV5 control viral particles did not show significant oxaluria reduction.

### DESCRIPTION OF THE INVENTION

### Definitions

A “nucleic acid construct” is herein understood to mean a man-made nucleic acid molecule resulting from the use of recombinant DNA technology. A nucleic acid construct is a nucleic acid molecule, either single- or double-stranded, which has been modified to contain segments of nucleic acids, which are combined and juxtaposed in a manner, which would not otherwise exist in nature. A nucleic acid construct usually is a “vector”, i.e. a nucleic acid molecule which may be used to deliver exogenously created nucleic acids, such as DNA into a host cell.

One type of nucleic acid construct is an “expression cassette” or “expression vector”. These terms refers to nucleotide sequences that are capable of effecting expression of a gene in a host cell or a host organism compatible with such sequences. Expression cassettes or expression vectors typically include at least suitable transcription regulatory sequences and optionally, 3′ transcription termination signals. Additional factors necessary or helpful in effecting expression may also be present, such as expression enhancer elements.

The term “homologous” when used to indicate the relation between a given (recombinant) nucleic acid or polypeptide molecule and a given host organism or host cell, is understood to mean that in nature the nucleic acid or polypeptide molecule is produced by a host cell or an organism of the same species. The term “heterologous” may be used to indicate that in nature the nucleic acid or polypeptide molecule is produced by a host cell or an organism of a different species.

As used herein, the term “operably linked” refers to a linkage of polynucleotide (or polypeptide) elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

“Expression control sequence” refers to a nucleic acid sequence that regulates the expression of a nucleotide sequence to which it is operably linked. An expression control sequence is “operably linked” to a nucleotide sequence when the expression control sequence controls and regulates the transcription and/or the translation of the nucleotide sequence. Thus, an expression control sequence can include promoters, enhancers, internal ribosome entry sites (IRES), transcription terminators, a start codon in front of a protein-encoding gene, splicing signal for introns, and stop codons. The term “expression control sequence” is intended to include, at a minimum, a sequence whose presence is designed to influence expression, and can also include additional advantageous components. For example, leader sequences and fusion partner sequences are expression control sequences. The term can also include the design of the nucleic acid sequence such that undesirable, potential initiation codons in and out of frame, are removed from the sequence. It can also include the design of the nucleic acid sequence such that undesirable potential splice sites are removed. It includes sequences or polyadenylation sequences (pA) which direct the addition of a polyA tail, i.e., a string of adenine residues at the 3′-end of a mRNA, sequences referred to as polyA sequences. It also can be designed to enhance mRNA stability. Expression control sequences which affect the transcription and translation stability, e.g., promoters, as well as sequences which effect the translation, e.g., Kozak sequences, are known in insect cells. Expression control sequences can be of such nature as to modulate the nucleotide sequence to which it is operably linked such that lower expression levels or higher expression levels are achieved.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter, including e.g. attenuators or enhancers, but also silencers.

A “constitutive” promoter is a promoter that is active in most tissues under most physiological and developmental conditions. An “inducible” promoter is a promoter that is physiologically or developmentally regulated, e.g. by the application of a chemical inducer. A “tissue specific” promoter is only active, substantially only active or predominantly active in specific types of tissues or cells.

A “3′ UTR” or “3′ non-translated sequence” (also often referred to as 3′ untranslated region, or 3′ end) refers to the nucleic acid sequence found downstream of the coding sequence of a gene, which comprises, for example, a transcription termination site and (in most, but not all eukaryotic mRNAs) a polyadenylation signal (such as e.g. AAUAAA or variants thereof). After termination of transcription, the mRNA transcript may be cleaved downstream of the polyadenylation signal and a poly(A) tail may be added, which is involved in the transport of the mRNA to the cytoplasm (where translation takes place).

The terms “substantially identical”, “substantial identity” or “essentially similar” or “essential similarity” means that two peptide or two nucleotide sequences, when optimally aligned, such as by the programs GAP or BESTFIT using default parameters, share at least a certain percentage of sequence identity as defined elsewhere herein. GAP uses the Needleman and Wunsch global alignment algorithm to align two sequences over their entire length, maximizing the number of matches and minimizes the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). It is clear than when RNA sequences are said to be essentially similar or have a certain degree of sequence identity with DNA sequences, thymine (T) in the DNA sequence is considered equal to uracil (U) in the RNA sequence. Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA or the open-source software Emboss for Windows (current version 2.7.1-07). Alternatively percent similarity or identity may be determined by searching against databases such as FASTA, BLAST, etc.

### DETAILED DESCRIPTION OF THE INVENTION

AGXT I340M (a variant rarely seen in the general population) results in a significantly higher activity than the most common major allele. Herein, it is shown that a single tail vein administration of an AAV gene therapy vector incorporating nucleic acids encoding AGXT with I340M result in sustained correction of the primary hyperoxaluria type I (PH1) phenotype in a mouse model, without evidence of liver damage or toxicity.

This AAV-based gene transfer approach resulted in liver expression of the human AGXT I340M sufficiently robust so as to overcome the potential problem posed by the continued oxalate production of non-transduced hepatocytes. Further, the expressed gene is correctly targeted to the peroxisome in the mouse hepatocyte, and it complements the deficit of the mouse Agxt1KO model.

Accordingly, the invention pertains to an AGXT therapeutic, preferably an AGXT I340M therapeutic. That is to say, the invention involves the recognition of important advantages that may be obtained through therapeutic treatments comprising the administration of therapeutics derived from the AGXT protein, such as the AGXT I340M protein, and nucleic acid sequences encoding the AGXT protein, such as the AGXT I340M protein.

In the context of this invention an AGXT therapeutic, including an AGXT I340M therapeutics, may include AGXT (including AGXT I340M) peptides, nucleic acid sequences coding therefore, cells expressing such peptides or nucleic acids, and derivatives of such peptides, wherein the said therapeutic typically ameliorates or treats disease when administered in prophylactically or therapeutically effective dosages.

AGXT therapeutics, including AGXT I340M therapeutics, of the invention include modifications, derivatives and analogs of AGXT (or AGXT I340M) peptides, and nucleic acids encoding such peptides. In some embodiments, the AGXT therapeutic, such as an AGXT I340M therapeutic, of the invention may be a peptide having a sequence of amino acids corresponding to the naturally-occurring wild-type AGXT peptide or the AGXT I340M peptide as set out in SEQ ID NO: 2.

The AGXT therapeutic, such as an AGXT I340M therapeutic, of the invention may include substantially purified compounds such as peptide fragments, modified peptide fragments, analogues or pharmacologically acceptable salts of AGXT (or AGXT I340M). Such compounds are collectively referred to herein as LPL S447X peptides. AGXT (or AGXT I340M) peptides may include homologs of the wild-type mature AGXT sequence (or homologs of the AGXT I340M sequence), including homologs from species other than homo sapiens (which may have veterinary applications).

AGXT (or AGXT I340M) peptides may include naturally occurring isoforms or genetic variants of wild type AGXT or AGXT I340M.

In one embodiment, an AGXT I340M therapeutic is an AGXT I340M protein comprising an amino acid sequence having at least about 50%, at least about 60%, at least about 70%, at least about 80%, at least about 85%, at least about 90%, at least about 95%, at least about 97%, at least about 98%, at least about 99% or at least about 100% sequence identity to SEQ ID NO: 2 when optimally aligned over their entire length using the GAP program, and wherein the AGXT I340M protein comprises a methionine at a position corresponding to position 340 in SEQ ID NO: 2. A “position corresponding to a certain position in SEQ ID NO: 2” (e.g. 340) is herein understood as a position corresponding to that position in SEQ ID NO: 2 in an AGXT amino acid sequence other than SEQ ID NO: 2, when optimally aligned with SEQ ID NO: 2 over the entire length of using the GAP program.

In one embodiment the AGXT I340M protein is a protein having alanine:glyoxylate aminotransferase (AGXT; EC 2.6.1.44) activity as may be assayed as described by Rumsby et al (Ann Clin Biochem. 1997 July; 34 (Pt 4):400-4). In a preferred embodiment, the AGXT I340M protein is a protein having an amino acid sequence wherein active site residues, residues involved in substrate-binding, co-factor (pyridoxal 5′-phosphate) binding and/or subunit interaction are conserved. Such residues may be derived by comparison of the amino acid sequences of SEQ ID NO: 2 with the 3D-crystal structure of the human and Anabaena AGXT proteins as has been described by Zhang et al. (2003, J Mol. Biol., 331(3):643-52) and Han et al. (2005, Proteins., 58(4):971-5), respectively), including identification of active site residues at positions 81 (S), 82 (G), 83 (H), 108 (W), 158 (S), 183 (D), 186 (A) 208 (Q) and 209 (K). According to Fold and Function Assignment System (FFAS; Jaroszewski L, Li W, Godzik A. 2002, Protein Sci, 11:1702-1713), the AGXT family has hundreds of homologous sequences in pro- and eukaryotic proteomes. Models for AGXT homologues can be accessed at http://www1.jcsg.org/cgi-bin/models/get_mor.pl?key_{—}17130350. Sequence alignments of SEQ ID NO: 2 with these known AGXT amino acid sequences will indicate conserved regions and amino acid positions, the conservation of which are important for structure and enzymatic activity. These regions and positions will tolerate no or only conservative amino acid substitutions. Amino acid substitutions outside of these regions and positions may be unlikely to greatly affect AGXT activity.

In a preferred embodiment, the AGXT I340M therapeutic is an AGXT I340M protein comprising amino acid substitions selected from the group consisting of a) a leucine at a position corresponding to position 11 in SEQ ID NO: 2; b) an arginine at a position corresponding to position 41 in SEQ ID NO: 2; c) an isoleucine at a position corresponding to position 152 in SEQ ID NO: 2; d) an arginine at a position corresponding to position 170 in SEQ ID NO: 2; e) a threonine at a position corresponding to position 244 in SEQ ID NO: 2; f) a threonine at a position corresponding to position 294 in SEQ ID NO: 2; g) an isoleucine at a position corresponding to position 326 in SEQ ID NO: 2; h) combinations of one or more of the substitutions amino acid b) to g); and, i) the combination of the substitutions a) and f). In a preferred embodiment, the AGXT I340M therapeutic is an AGXT I340M protein selected from the group consisting of: a) an AGXT I340M protein comprising the amino acid sequence of SEQ ID NO: 2; and, b) an AGXT I340M protein comprising the amino acid sequence of SEQ ID NO: 2 and comprising amino acid substitions as defined in a) to i) above.

In another embodiment, a AGXT I340M therapeutic is a nucleic acid molecule comprising an nucleotide sequence encoding the AGXT I340M protein as defined herein above, or a virion of a viral gene therapy vector comprising the nucleic acid molecule.

In a preferred embodiment of the invention, a nucleotide sequence coding for the AGXT I340M protein has an improved codon usage bias for the human cell as compared to naturally occurring nucleotide sequence coding for the transferase.

The adaptiveness of a nucleotide sequence encoding the AGXT I340M protein to the codon usage of human cells may be expressed as codon adaptation index (CAI). A codon adaptation index is herein defined as a measurement of the relative adaptiveness of the codon usage of a gene towards the codon usage of highly expressed human genes. The relative adaptiveness (w) of each codon is the ratio of the usage of each codon, to that of the most abundant codon for the same amino acid. The CAI is defined as the geometric mean of these relative adaptiveness values. Non-synonymous codons and termination codons (dependent on genetic code) are excluded. CAI values range from 0 to 1, with higher values indicating a higher proportion of the most abundant codons (see Sharp and Li, 1987, Nucleic Acids Research 15: 1281-1295; also see: Kim et al., Gene. 1997, 199:293-301; zur Megede et al., Journal of Virology, 2000, 74: 2628-2635). Preferably, a nucleotide sequence encoding the AGXT I340M protein has a CAI of at least about 0.8, at least about 0.85, at least about 0.90, at least about 0.92, at least about 0.94, at least about 0.95, at least about 0.96 or at least about 0.97. The coding sequence may further be adapted for improved expression in the insect host cell by methods described in WO 2004/059556, and by modifying the CpG content of the coding sequence as described in WO 2006/015789. It is understood that such further adaptations may cause that not all codons in the nucleotide sequence coding for the common amino acid sequence in the first nucleotide sequence are common codons.

In one embodiment of invention, a nucleotide sequence encoding the AGXT I340M protein has the nucleotide sequence of SEQ ID NO: 1.

The invention also pertains to a nucleic acid construct comprising a nucleotide sequence encoding the AGXT I340M protein of the invention as herein defined above. In the nucleic acid construct the nucleotide sequence encoding the AGXT I340M protein preferably is operably linked to a mammalian cell-compatible expression control sequence, e.g., a promoter, preferably a mammalian cell-compatible promoter. Many such promoters are known in the art (see Sambrook and Russel, 2001, supra). Constitutive promoters that are broadly expressed in many cell-types, such as the CMV promoter may be used. However, more preferred will be promoters that are inducible, tissue-specific, cell-type-specific, or cell cycle-specific. In a preferred embodiment a nucleotide sequence encoding the AGXT I340M protein is operably linked to a liver-specific promoter. Liver-specific promoters are particularly preferred for use in conjunction the non-erythroid deaminase.

Preferably, in a construct of the invention expression control sequences for liver-specific expression are e.g. selected from the group consisting of an α1-anti-trypsin (AAT) promoter, a thyroid hormone-binding globulin promoter, an albumin promoter, a thyroxin-binding globulin (TBG) promoter, an Hepatic Control Region (HCR)-ApoCII hybrid promoter, an HCR-hAAT hybrid promoter, an AAT promoter combined with the mouse albumin gene enhancer (Ealb) element and an apolipoprotein E promoter. Other examples include the E2F promoter for tumour-selective, and, in particular, neurological cell tumour-selective expression (Parr et al., 1997, Nat. Med. 3:1145-9) or the IL-2 promoter for use in mononuclear blood cells (Hagenbaugh et al., 1997, J Exp Med; 185: 2101-10). In a particularly preferred embodiment of the invention, the promoter may have the sequence of SEQ ID NO: 3. In another embodiment of the nucleic acid construct, a nucleotide sequence encoding the AGXT I340M protein is operably linked to a promoter for expression in human cells that is not a promoter from an AGXT gene, for example not a promoter from a human AGXT gene.

In a further preferred embodiment, the nucleic acid construct of the invention comprises a 3′UTR (or 3′ non-translated sequence) downstream of the nucleotide sequence encoding the AGXT I340M protein. Suitable 3′UTR sequences are available to the skilled person. They may be derived from any mammalian and preferably human gene and will usually comprise a transcription termination site and a polyadenylation signal (such as e.g. AAUAAA or variants thereof). In a particularly preferred embodiment the nucleic acid construct comprises a 3′UTR derived from the human porphobilinogen deaminase gene such as e.g. SEQ ID NO: 4.

In another preferred embodiment of the nucleic acid construct of the invention, the expression control sequence that is operably linked to the nucleotide sequence encoding the AGXT I340M protein, is preceded upstream by a polyA insulator to terminate run-through transcription from possible upstream transcription units. A 3′UTR as described above and preferably at least comprising a transcription termination sequence may be used for this purpose. A preferred polyA insulator is a synthetic polyA insulator having the sequence of SEQ ID NO: 5.

In one preferred embodiment, the nucleic acid construct of the invention comprises a Kozak consensus sequence around the initiation codon of the nucleotide sequence encoding the AGXT I340M protein.

The Kozak consensus sequence is herein defined as GCCRCC(AUG)A (SEQ ID NO: 6), wherein R is a purine (i.e. A, adenosine or G, guanosine) and wherein (AUG) stands for the initiation codon of the AGXT I340M protein-coding sequence. In a preferred embodiment, the Kozak consensus sequence may be preceded by another GCC triplet.

The invention also provides a nucleic acid construct comprising a nucleotide sequence encoding the AGXT I340M protein that is operably linked to an expression control sequence as defined herein above, wherein the construct is an expression vector that is suitable for gene therapy of mammals, preferably gene therapy of humans. A preferred nucleic acid construct according to the invention is a viral gene therapy vector. Viral gene therapy vectors are well known in the art and e.g. include vectors based on an adenovirus, a parvovirus such as an adeno-associated virus (AAV), a herpes virus, a pox virus and a retrovirus. A preferred viral gene therapy vector is an AAV, adenoviral or a lentiviral vector.

Particularly preferred gene therapy vectors in the context of the present invention are parvoviral vectors. Thus, in this preferred aspect the invention relates the use of animal parvoviruses, in particular dependoviruses such as infectious human or simian AAV, and the components thereof (e.g., an animal parvovirus genome) for use as vectors for introduction and/or expression of the nucleotide sequences encoding a AGXT I340M protein in mammalian cells.

Viruses of the Parvoviridae family are small DNA animal viruses. The family Parvoviridae may be divided between two subfamilies: the Parvovirinae, which infect vertebrates, and the Densovirinae, which infect insects. Members of the subfamily Parvovirinae are herein referred to as the parvoviruses and include the genus Dependovirus. As may be deduced from the name of their genus, members of the Dependovirus are unique in that they usually require coinfection with a helper virus such as adenovirus or herpes virus for productive infection in cell culture. The genus Dependovirus includes AAV, which normally infects humans (e.g., serotypes 2, 3A, 3B, 5, and 6) or primates (e.g., serotypes 1 and 4, which are thought to have been originated from monkeys, but also infect humans), and related viruses that infect other warm-blooded animals (e.g., bovine, canine, equine, and ovine adeno-associated viruses).

Further information on AAV serotypes and on strategies for engineering hybrid AAV vectors derived from AAV serotypes is described in Wu et al. (2006, Molecular Therapy 14:316-327). For convenience the present invention is further exemplified and described herein by reference to AAV. It is however understood that the invention is not limited to AAV but may equally be applied to hybrid AAV vectors derived from two or more different AAV serotypes and to other parvoviruses and hybrids thereof.

The genomic organization of all known AAV serotypes is very similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins (VP1, -2 and -3) form the capsid. The terminal 145 nt are self-complementary and are organized so that an energetically stable intramolecular duplex forming a T-shaped hairpin may be formed. These hairpin structures function as an origin for viral DNA replication, serving as primers for the cellular DNA polymerase complex. Following wtAAV infection in mammalian cells the Rep genes (i.e. Rep78 and Rep52) are expressed from the P5 promoter and the P19 promoter, respectively and both Rep proteins have a function in the replication of the viral genome. A splicing event in the Rep ORF results in the expression of actually four Rep proteins (i.e. Rep78, Rep68, Rep52 and Rep40). However, it has been shown that the unspliced mRNA, encoding Rep78 and Rep52 proteins, in mammalian cells are sufficient for AAV vector production. Also in insect cells the Rep78 and Rep52 proteins suffice for AAV vector production.

A “recombinant parvoviral or AAV vector” (or “rAAV vector”) herein refers to a vector comprising one or more polynucleotide sequences of interest, genes of interest or “transgenes” that are flanked by at least one parvoviral or AAV inverted terminal repeat sequences (ITRs). Such rAAV vectors can be replicated and packaged into infectious viral particles when present in an insect host cell that is expressing AAV rep and cap gene products (i.e. AAV Rep and Cap proteins). When an rAAV vector is incorporated into a larger nucleic acid construct (e.g. in a chromosome or in another vector such as a plasmid or baculovirus used for cloning or transfection), then the rAAV vector is typically referred to as a “pro-vector” which can be “rescued” by replication and encapsidation in the presence of AAV packaging functions and necessary helper functions.

Thus, the invention relates to a nucleic acid construct comprising a nucleotide sequence encoding an AGXT I340M protein as herein defined above, wherein the nucleic acid construct is a recombinant parvoviral or AAV vector and thus comprises at least one parvoviral or AAV ITR. Preferably, in the nucleic acid construct the nucleotide sequence encoding the AGXT I340M protein is flanked by parvoviral or AAV ITRs on either side. Any parvoviral or AAV ITR may be used in the constructs of the invention, including ITRs from AAV1, AAV2, AAV4, and/or AAV5. ITRs of AAV2 are most preferred. Examples of preferred ITR sequences for use in preferred nucleic acid constructs of the invention are given SEQ ID NO: 7 (left or upstream ITR) and SEQ ID NO: 8 (right or downstream ITR).

AAV is able to infect a number of mammalian cells. See, e.g., Tratschin et al. (1985, Mol. Cell. Biol. 5:3251-3260) and Grimm et al. (1999, Hum. Gene Ther. 10:2445-2450). However, the cellular tropism of AAV differs among serotypes. See, e.g., Davidson et al. (2000, Proc. Natl. Acad. Sci. USA, 97:3428-3432), who discuss differences among AAV2, AAV4, and AAV5 with respect to mammalian CNS cell tropism and transduction efficiency and see Goncalves, 2005, Virol J. 2(1):43 who discusses approaches to modification of AAV tropism. For transduction of liver cells rAAV virions with AAV 1, AAV8 and AAV5 capsid proteins are preferred (Nathwani et al., 2007, Blood 109(4): 1414-1421; Kitajima et al., 2006, Atherosclerosis 186(1):65-73), of which is rAAV virions with AAV5 or 8 capsid proteins are most preferred.

AAV sequences that may be used in the present invention for the production of recombinant AAV vectors in insect cells can be derived from the genome of any AAV serotype. Generally, the AAV serotypes have genomic sequences of significant homology at the amino acid and the nucleic acid levels, provide an identical set of genetic functions, produce virions which are essentially physically and functionally equivalent, and replicate and assemble by practically identical mechanisms. For the genomic sequence of the various AAV serotypes and an overview of the genomic similarities see e.g. GenBank Accession number U89790; GenBank Accession number J01901; GenBank Accession number AF043303; GenBank Accession number AF085716; Chlorini et al. (1997, J. Vir. 71: 6823-33); Srivastava et al. (1983, J. Vir. 45:555-64); Chlorini et al. (1999, J. Vir. 73:1309-1319); Rutledge et al. (1998, J. Vir. 72:309-319); and Wu et al. (2000, J. Vir. 74: 8635-47). AAV serotypes 1, 2, 3, 4 and 5 are preferred source of AAV nucleotide sequences for use in the context of the present invention. Preferably the AAV ITR sequences for use in the context of the present invention are derived from AAV1, AAV2, and/or AAV4. Likewise, the Rep (Rep78/68 and Rep52/40) coding sequences are preferably derived from AAV1, AAV2, and/or AAV4. The sequences coding for the VP1, VP2, and VP3 capsid proteins for use in the context of the present invention may however be taken from any of the known 42 serotypes, more preferably from AAV1, AAV2, AAV3, AAV4, AAV5, AAV6, AAV7, AAV8 or AAV9 or newly developed AAV-like particles obtained by e.g. capsid shuffling techniques and AAV capsid libraries.

AAV Rep and ITR sequences are particularly conserved among most serotypes. The Rep78 proteins of various AAV serotypes are e.g. more than 89% identical and the total nucleotide sequence identity at the genome level between AAV2, AAV3A, AAV3B, and AAV6 is around 82% (Bantel-Schaal et al., 1999, J. Virol., 73(2):939-947). Moreover, the Rep sequences and ITRs of many AAV serotypes are known to efficiently cross-complement (i.e., functionally substitute) corresponding sequences from other serotypes in production of AAV particles in mammalian cells. US2003148506 reports that AAV Rep and ITR sequences also efficiently cross-complement other AAV Rep and ITR sequences in insect cells.

The AAV VP proteins are known to determine the cellular tropism of the AAV virion. The VP protein-encoding sequences are significantly less conserved than Rep proteins and genes among different AAV serotypes. The ability of Rep and ITR sequences to cross-complement corresponding sequences of other serotypes allows for the production of pseudotyped rAAV particles comprising the capsid proteins of one serotype (e.g., AAV5) and the Rep and/or ITR sequences of another AAV serotype (e.g., AAV2). Such pseudotyped rAAV particles are a part of the present invention.

Modified “AAV” sequences also can be used in the context of the present invention, e.g. for the production of rAAV vectors in insect cells. Such modified sequences e.g. include sequences having at least about 70%, at least about 75%, at least about 80%, at least about 85%, at least about 90%, at least about 95%, or more nucleotide and/or amino acid sequence identity (e.g., a sequence having about 75-99% nucleotide sequence identity) to an AAV1, AAV2, AAV3, AAV4, AAV5, AAV6, AAV7, AAV8 or AAV9 ITR, Rep, or VP can be used in place of wild-type AAV ITR, Rep, or VP sequences.

Although similar to other AAV serotypes in many respects, AAV5 differs from other human and simian AAV serotypes more than other known human and simian serotypes. In view thereof, the production of rAAV5 can differ from production of other serotypes in insect cells. Where methods of the invention are employed to produce rAAV5, it is preferred that one or more constructs comprising, collectively in the case of more than one construct, a nucleotide sequence comprising an AAV5 ITR, a nucleotide sequence comprises an AAV5 Rep coding sequence (i.e. a nucleotide sequence comprises an AAV5 Rep78). Such ITR and Rep sequences can be modified as desired to obtain efficient production of rAAV5 or pseudotyped rAAV5 vectors in insect cells. E.g., the start codon of the Rep sequences can be modified, VP splice sites can be modified or eliminated, and/or the VP1 start codon and nearby nucleotides can be modified to improve the production of rAAV5 vectors in the insect cell.

If an adenoviral vector is used in the invention, a preferred adenoviral vector may be modified to reduce the host response as reviewed by Russell (2000, J. Gen. Viral. 81: 2573-2604), or as described in US20080008690 and by Zaldumbide and Hoeben (Gene Therapy 2008:239-246).

The invention thus relates to a parvoviral virion comprising a nucleic acid construct as herein defined above, and parvoviral capsid protein as defined herein above.

The invention also provides a method for producing a recombinant parvoviral (rAAV) virion (comprising a recombinant parvoviral (rAAV) vector as defined above) in an insect cell. Preferably, the method comprises the steps of (a) culturing an insect cell as defined in herein above under conditions such that recombinant parvoviral (rAAV) vector is produced; and, (b) recovery of the recombinant parvoviral (rAAV) vector. It is understood here that the recombinant parvoviral (rAAV) vector produced in the method preferably is an infectious parvoviral or AAV virion that comprise the recombinant parvoviral (rAAV) vector nucleic acids. Growing conditions for insect cells in culture, and production of heterologous products in insect cells in culture are well-known in the art and described e.g. in the above cited references on molecular engineering of insects cells. Preferred methods and constructs for the production of rAAV virions of the invention are disclosed in e.g. WO2007/046703 and WO2007/148971.

Preferably the methods for producing recombinant parvoviral virions further comprises the step of affinity-purification of the (virions comprising the) recombinant parvoviral (rAAV) vector using an anti-AAV antibody, preferably an immobilised antibody. The anti-AAV antibody preferably is a monoclonal antibody. A particularly suitable antibody is a single chain camelid antibody or a fragment thereof as e.g. obtainable from camels or llamas (see e.g. Muyldermans, 2001, Biotechnol. 74: 277-302). The antibody for affinity-purification of rAAV preferably is an antibody that specifically binds an epitope on a AAV capsid protein, whereby preferably the epitope is an epitope that is present on capsid protein of more than one AAV serotype. E.g. the antibody may be raised or selected on the basis of specific binding to AAV2 capsid but at the same time also it may also specifically bind to AAV 1, AAV3 and AAV5 capsids.

The invention further pertains to an AGXT I340M therapeutic for use as a medicament or for use in a method of treatment. The AGXT I340M therapeutic for use as a medicament can be an AGXT I340M protein or nucleic acid molecule as herein defined above, or a virion of a gene therapy vector, such as e.g. a parvoviral virion as defined above.

Also, the invention pertains to an AGXT I340M therapeutic as herein defined for use in the treatment of an AGXT-responsive condition.

An AGXT-responsive condition can be a condition caused by a deficiency (for example a partial deficiency or complete deficiency (or substantially complete deficiency)) in alanine:glyoxylate aminotransferase. A deficiency in alanine:glyoxylate aminotransferase is understood to include absence (partial, complete or substantially complete) or subcellular mislocation of the alanine:glyoxylate aminotransferase. AGXT deficiency leads to a disease termed primary hyperoxaluria type I, which is also referred to as oxalosis I, glycolic aciduria, alanine-glyoxylate aminotransferase deficiency, and peroxisomal and/or hepatic alanine-glyoxylate aminotransferase deficiency. Accordingly, the invention pertains to treatment of primary hyperoxaluria type I. Treatment according to the invention includes treatment of one or more symptoms, such as amelioration thereof, of such an indication.

The invention also pertains to a pharmaceutical composition comprising an AGXT I340M therapeutic, a nucleic acid construct or a parvoviral virion as herein defined above. The pharmaceutical composition further preferably comprises a pharmaceutically acceptable carrier. Any suitable pharmaceutically acceptable carrier or excipient can be used in the present compositions (See e.g., Remington: The Science and Practice of Pharmacy, Alfonso R. Gennaro (Editor) Mack Publishing Company, April 1997).

Preferred pharmaceutical forms would be in combination with sterile saline, dextrose solution, or buffered solution, or other pharmaceutically acceptable sterile fluids. Alternatively, a solid carrier, may be used such as, for example, microcarrier beads.

Also, the invention provides a method for treating an AGXT-responsive condition wherein the method comprises the step of administering an effective amount of an AGXT I340M therapeutic, a nucleic acid construct, a parvoviral virion or a pharmaceutical composition as defined herein to a subject with the AGXT-responsive condition. The AGXT-responsive condition can be a deficiency in alanine:glyoxylate aminotransferase such as e.g. primary hyperoxaluria type I.

In addition, the invention relation to use of an AGXT I340M therapeutic, a nucleic acid construct, a parvoviral virion or a pharmaceutical composition as defined herein in the manufacture of a medicament for use in the treatment of an AGXT-responsive condition.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

All patent and literature references cited in the present specification are hereby incorporated by reference in their entirety.

The following examples are offered for illustrative purposes only, and are not intended to limit the scope of the present invention in any way.

### EXAMPLES

### 1. Example 1

The human AGXT cDNA wild type and several major and minor alleles and combination thereof were expressed with His-tags in E. coli (Top 10, Invitrogen). Expressed AGXT proteins were purified by metal-affinity chromatography according to standard procedures.

Purified protein was subject to SDS/PAGE analysis and Alanine-glyoxylate aminotransferase activity assay (Rumsby et al., Ann Clin Biochem. 1997 July; 34 (Pt 4):400-4.). Mean activity values (umoles/h/g total protein) of the AGXT proteins as expressed in E. coli are given in Table 1.

Table 1 Mean activity values (umoles/h/g total protein) of the AGXT proteins as expressed in E. coli. pTrcHis=empty vector; pTrcHisAGXT*WT=wild type AGXT (Genbank Acc. No. NM_{—}000030); T indicates the I244T allele; M indicates the I340M allele; *L indicates the P11L allele; and R indicates the G170R allele. Combinations of these letter indicate proteins with the corresponding amino acid substitutions in one protein.

| CONSTRUCT | MEAN ACTIVITY | Standard Deviation |
|---|---|---|
| pTrcHis (Top10) | 0.370 | 0.169 |
| pTrcHisAGXT*WT | 36.351 | 11.735 |
| pTrcHisAGXT*T | 26.517 | 3.636 |
| pTrcHisAGXT*M | 45.920 | 8.399 |
| pTrcHisAGXT*L | 12.489 | 2.694 |
| pTrcHisAGXT*LM | 21.255 | 4.016 |
| pTrcHisAGXT*LR | 2.122 | 0.534 |
| pTrcHisAGXT*LRM | 8.617 | 1.289 |
| pTrcHisAGXT*LT | 0.923 | 0.319 |
| pTrcHisAGXT*LTM | 3.562 | 0.811 |

P11L is enough to promote a significant decrease in activity. Thus, the effect of the mutations depends significantly on the allelic background. Both G170R and I244T, in the minor haplotype, result in significant reduction of activity. The polymorphism I340M, when present in combination with either mutation, results in better residual activity. The expression of AGXT wild type with I340M (a variant rarely seen in the general population) results in a significantly higher activity than the most common major allele.

### 2. Example 2

### 2.1 Materials and Methods

### 2.1.1 Animal Experiments, Urine and Blood Analyses

All animal experiments were approved by the institutional animal experimentation ethics committee, and carried out according to the Spanish and European law. B6;129SvAgxt^{tm1U11} mice were genotyped as described [Salido et al. PNAS 2006; 103(48):18249-54. PMID: 17110443], bred and maintained in a pathogen-free facility, with free access to standard chow (A04, Safe, France) and water. Mice were placed in metabolic cages for single mouse (model 3600M, Tecniplast) and allowed to get acclimatized and used to a ˜4 g/day powdered, oxalate-free diet (with <0.4 μmol/g oxalate, 0.5% Ca²⁺, and 0.25% Mg²⁺ (Harlan TD#01699, Indianapolis, Ind., USA,) during 3 days before the start of urine collection. Two 24 hr urine collections were performed each week in narrow tubes containing 50 μl 6 N HCl. The oxalate oxidase assay (http://www.greiner-diagnostic.com) was used to measure urine oxalate, while the Jaffe alkaline picrate test was used to measure urine creatinine. Twenty-four-hour urine samples that were <1 ml for 3 month old mice were typically seen in cages with signs of incomplete urine collection, and were excluded from the study. Similarly, samples with food or fecal contamination were excluded. At the end of the study, the mice were sacrificed, and blood was collected for biochemical analysis. Serum creatinine, BUN, GOT and GPT were measured in an autoanalyzer (A25, Biosystems, Spain). Serum samples were also used, diluted 1:100 in PBS, in Western blot analysis of recombinant human AGXT protein, to check for the presence of anti-AGXT mouse antibodies.

Liver samples were harvested for histology, enzyme assay, and northern and western blot analyses. Kidney samples were collected for histological analysis. Five males were selected for a more extensive histological study that included brain, lung, heart, stomach, liver, spleen, pancreas, kidney, testis and seminal vesicle. DNA and protein were also extracted from these tissues.

For gene therapy experiments, 12-16 week old mice were restrained and injected into the tail vein using a 1 ml syringe with 27G needle. AAV particles were dissolved in 5% sucrose-PBS solution.

### 2.1.2 Histological Analysis

Tissues were fixed in 4% buffered paraformaldehyde and either embedded in paraffin or cryoprotected in 20% sucrose and snap-frozen in liquid nitrogen. Hematoxylin and eosin staining was done in all tissues, and calcium oxalate staining (Yasue, 1969 Acta Histochemica et Cytochemica 2:83-95) was performed on kidney sections. Kidney sections from a PH1 patient with nephrocalcinosis were used as positive control for the calcium oxalate staining. To measure the deposits, two random sections stained for CaOx were digitally imaged and processed with image analysis software (ImagePro). The threshold for positive areas was established automatically, and the area of the deposits was expressed as a percentage of the total digitized area.

### 2.1.3 AGT Enzyme Assay, Western Blot and Immunohistochemistry

AGT activity and western blot analyses were determined as described [Salido et al. PNAS 2006; 103(48): 18249-54. PMID: 17110443]. Immunohistochemistry was performed on frozen sections, incubating with 1:5000 rabbit anti-human AGXT antibody (a gift from Dr. C. Danpure) for 2 hrs, followed by 5 min. PBS washes, 3 times, and HRP-conjugated anti-rabbit serum (Dako), for 30 min. After another 3 PBS washes, a DAB-H2O2 solution was used as chromogen, and some sections were counterstained with hematoxylin. Control sections were also stained with 1:5000 anti-GS antibody (Santa Cruz Biotechnologies) to label perivenular zones.

### 2.1.4 Recombinant AAV Construction, Production and DNA Analysis

The AAV plasmids used in this study contain the expression cassette flanked by two ITRs from the AAV2 and an appropriate stuffier sequence to adjust genome size to the optimal packaging capacity described for AAV. The expression cassette has the following elements: the 5′ITR from AAV2, a liver-specific promoter EalbAATp with regulatory sequences from the albumin enhancer (Kramer et al., 2003, Mol. Ther. 7(3):375-85), either human AGXT cDNA (encoding the AGXT I340M variant—SEQ ID NO: 2) or enhanced GFP cDNA, the bovine growth hormone polyadenylation sequence, a woodchuck hepatitis virus posttranscriptional regulatory element (WPRE), and the 3′ITR from AAV2. Similar expression cassettes were also made without the WPRE sequence. The four AAV plasmids used in this study are named ssAAV-EalbAAT-AGT-polyA-WPRE, ssAAV-EalbAAT-AGT-polyA (expressing the therapeutic gene), ssAAV-EalbAAT-GFP-polyA-WPRE and ssAAV-EalbAAT-GFP-polyA (expressing the reporter gene eGFP).

AAV2/8 vectors were produced by calcium phosphate-mediated co-transfection in 293 cells of three different plasmids pAdDeltaF6, p5E18-VD2/8 and the therapeutic (AAV-polyA-EalbAAT-AGT-WPRE) or reporter gene (AAV-polyA-EalbAAT-GFP-WPRE), (Hermens et al, 1999, Human Gene Therapy, 10:1885-91 and Gao et al 2002, Proc Natl Acad Sci USA 99:11854-9). Similarly, AAV2/5 vectors were produced using p5E18-VD2/5 instead. Briefly, 293 cells were co-transfected with pAdDeltaF6, either p5E18-VD2/8 or p5E18-VD2/5, and target vector by calcium phosphate and the virus was harvested by freeze thawing of the cells, 48 h after transfection. The virus was purified by ion exchange column chromatography and iodixanol gradient centrifugation followed by filtration and further concentration against phosphate-buffered saline (PBS)-5% sucrose. Virus titers in terms of genome copies/ml were determined by Q-PCR performed in triplicate, using TaqMan (AppliedBiosystems) protocols, and primers pr300fw (5′-CCCTGTTTGCTCCTCCGATAA-3′) and pr301rv (5′-GTCCGTATTTAAGCAGTGGATCCA-3′), which amplify a 95 bp fragment of the hAAT promoter region. Protein composition and purity was determined by sodium dodecyl sulfate-polyacrylamide gel electrophoresis (SDS-PAGE).

The number of AAV vector genomes per cell from different tissues was determined by real time PCR analysis with primers corresponding to the AGXT and WPRE sequences. Genomic DNA from different mouse tissues (liver, heart, brain, kidney, lung, spleen, testis and seminal vesicle) was isolated by proteinase K treatment and phenol extraction. About 100 ng of genomic DNA was used as template for PCR. Dilutions of the rAAV vector plasmid were used to generate a standard curve for determination of vector genome copies.

### 2.2 Results and Discussion

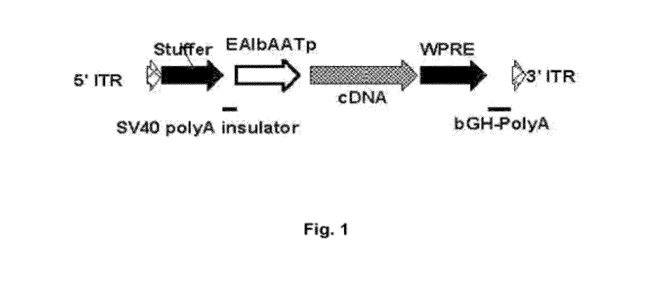

rAAV vectors were constructed by inserting the human AGXT-cDNA into an AAV2 vector plasmid, under the control of a hybrid EalbAAT liver-specific promoter. cDNAs were followed by a bGH polyadenylation signal and, in some constructs, WPRE post-transcriptional regulatory element (FIG. 1).

The expression cassette was flanked by inverted terminal repeats from AAV2. AAV2/8 and AAV2/5 vectors were produced by calcium phosphate-mediated co-transfection in 293 cells of three different plasmids pAdDeltaF6, either p5E18-VD2/8 or p5E18-VD2/5 and the therapeutic (either AAV-EalbAAT-AGT-polyA-WPRE or AAV-EalbAAT-AGT-polyA) or reporter gene (either AAV-EalbAAT-GFP-polyA-WPRE or AAV-EalbAAT-GFP-polyA), (Hermens et al, 1999 and Gao et al, 2002 Proc Natl Acad Sci USA 99:11854-9).

In a first set of experiments, we used AAV8 vectors at doses of 5×10¹² viral particles/kg body weight, aimed to transduce most of the hepatocytes, in male Agxt1KO mice (n=10 animals per group). Our goal was a sustained correction of hyperoxaluria in a 50 day follow-up. In addition, we wanted to test the functional reserve provided by gene transfer after a challenge with ethylene glycol, a glyoxylate precursor.

Mice were placed in metabolic cages, fed oxalate-free diet and water ad-libitum, and allowed to adjust for 3 days. Basal oxalate excretion was measured in 24 hr. urine, collected in acidified tubes, using an oxalate oxidase-based kit. Creatinine concentration was also measured by the alkaline picric acid method. Mean daily oxalate excretion was 2.06±0.74 moles/24 hrs. Purified, high titer (1.7−6.3×10¹¹ viral particles/ml) preparations of rAAV2-AGXT-2/8 vectors were administered to adult Agxt1KO mice, 12-16 weeks old, by single tail vein injections at a dose of 5×10¹² viral particles/kg body weight, in 0.2 ml PBS-sucrose. An additional group of 5 Agxt^{+/+} males, not injected with any vector, were used as normal controls.

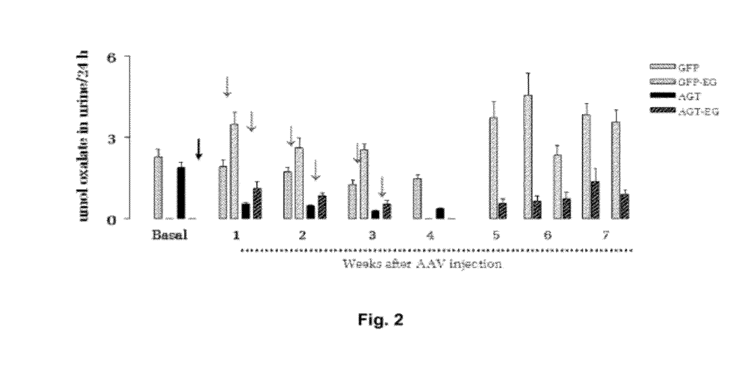

Urine oxalate excretion was followed during a total 50 day observation period, FIG. 2 shows the mean oxalate excretion for animals injected with either the human AGXT cDNA expressing vector or the GFP expressing control. During the first two weeks of the study, mice were challenged with 0.25 ml of 0.5M ethylene glycol (EG) in three occasions, by gavage, to follow the response to a discrete overload of the glyoxylate pathway. Both basal oxaluria and the increase in oxalate levels following the administration of the glyoxylate precursor (EG) were significantly lower in the treated group, compared with the controls, as evidenced after only 6 days post-injection, bringing urine oxalate levels in the treated animals 4 weeks after injection down to the range normally observed in wild type mice (0.37±0.11 vs. 0.31±0.13 μmoles/24 hr, respectively, p=0.0.28).

The response to the administration of 0.25 ml of 0.5M ethylene glycol was significantly blunted in mice treated with AGXT-AAV8 compared to those receiving GFP-AAV8 (0.34±0.37 μmol/24 hrs vs. 1.24±0.57 μmol/24 hrs, respectively, p=0.001). After 4 weeks, 0.5% EG was included in the drinking water, and the increase in oxalate excretion was significantly smaller in AGXT-AAV8 treated animals than in controls injected with GFP-AAV8 (increase of 0.18±0.34 vs. 2.11±1.31 moles/24 hr, respectively, p<0.001) and even smaller than in wild type mice subjected to the same challenge (0.50±0.21 moles/24 hr, p=0.005). These data are consistent with AGXT-AAV8 injection accounting for a larger functional reserve in treated mice than even wild type animals. During the last two weeks of the experiment, mice were subjected to 0.7% EG in drinking water, which resulted in significantly smaller increases of oxaluria in AGXT-AAV8 treated animals than in controls injected with GFP-AAV8 (increase of 0.54±0.56 vs. 2.46±0.7 μmoles/24 hr, respectively, p=0.000), a response not significantly different from the one observed in wild type controls drinking 0.7% ethylene glycol (increase of 0.92±0.33, p=0.06).

During the study period, three animals in the control group died with pathological finding of nephrocalcinosis (see below). None of the AAV8-AGXT treated mice died. At the end of the seventh week of study, mice were sacrificed and tissues and blood were collected. All kidneys from the AAV8-GFP injected mice, but none from the mice that received AAV8-AGXT, exhibited some degree of nephrocalcinosis at the end of the study. Four mice showed few calcium oxalate (CaOx) deposits in the medullary region only; three mice presented moderate CaOx deposits in the cortex and medulla; and in the three mice that died there was severe and widespread nephrocalcinosis. Plasma could not be obtained from the mice that died prematurely, but blood urea nitrogen (BUN) concentrations were significantly elevated among the remaining seven AAV8-BUN treated mice compared to those that received AAV8-AGXT (26.7±9.9 vs 19.1±1.3 mg/dl, respectively, p=0.04).

Hepatic AGT enzyme activity and protein, by western blot and immunohistochemistry, were determined in all sacrificed animal at the end of the seventh week after injection. AGT activity was significantly higher in Agxt^{-/-} mice injected with AGXT-AAV2/8 than in the GFP-AAV2/8 controls (24.9±10.6 vs. 4.1±1.5 nmole/min*mg protein, p=0.002). Significant residual activity is present in liver extracts of Agxt^{-/-} animals, mainly with alanine as a substrate (AGT activity), but also when serine was used as a substrate (SGT activity) [Salido et al. “Alanine-glyoxylate aminotransferase-deficient mice, a model for primary hyperoxaluria that responds to adenoviral gene transfer.” Proc. Natl. Acad. Sci. U.S.A 103.48 (2006): 18249-54].

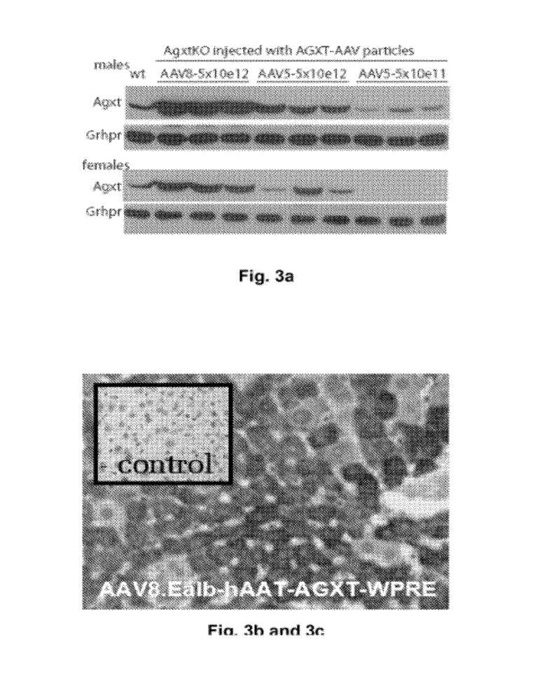

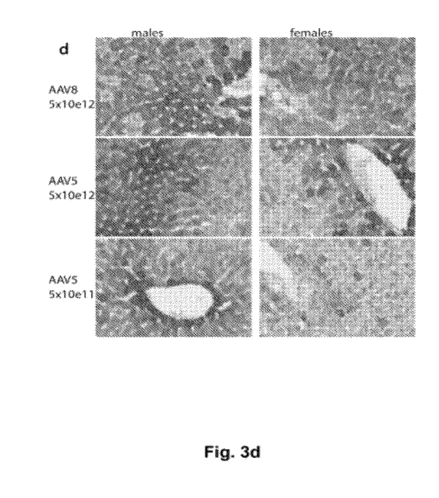

Western analysis of AGT protein in liver, as shown in FIG. 3a, revealed robust AGT expression in rAAV-treated mice (lanes 2 to 10) compared to wild-type (lane 1) while no protein could be detected in control Agxt1KO mice treated with GFP-AAV2/8.

Transduction rates and expression of AGXT-AAV2/8 in different tissues of treated animals was determined by western blot. Brain, lung, spleen, kidney, seminal vesicles and testicles did not contain detectable AGT protein, but heart samples consistently showed low levels of AGT protein, evident after longer exposures of film (data not shown). The presence of AGXT-AAV2/8 DNA in various tissues was evaluated by PCR, using primers annealing to the 3′ region of AGXT coding sequence and the WPRE sequence. Standard curves were made with AGXT-AAV8 and mouse interleukin 2 (mIL2) plasmids, and variations in the amount of template DNA were corrected by running parallel amplifications of the mIL2 gene. In livers of AGXT-AAV8 treated mice, we found 899±74 vector copies per diploid genome with the real-time PCR analysis. The estimation of vector copy number per mouse genome in other tissues were: 4.4±0.8 (kidney), 1.6±0.6 (lung), 0.7±0.1 (spleen), 1.3±0.1 (testis), and 3.3±0.6 (heart). Thus, two months after injection of 5×10¹² viral particles per mouse, close to a thousand rAAV copies per transduced hepatocyte are detected, similar to what others have reported for this type of vectors [Nakai H, Fuess S, Storm T A, Muramatsu S, Nara Y, Kay M A: Unrestricted hepatocyte transduction with adeno-associated virus serotype 8 vectors in mice. J Viral. 2005 79(1):214-24.PMID: 15596817].

By immunohistochemistry, using rabbit anti-AGT antibody, striking differences could be seen between livers from Agxt1KO mice treated with either AGXT-AAV2/8 or GFP-AAV2/8 (FIG. 3b). Indeed, most of the hepatocytes from mice injected with AGXT-AAV2/8 particles showed some degree of immunostaining, with a characteristic cytoplasmic granular pattern (FIG. 3c) while no AGT staining was observed in control mice injected with eGFP-AAV2/8, which, conversely, showed abundant green fluorescence in liver tissue sections under the UV microscope (not shown).

Primary cultures of isolated hepatocytes, performed in one mouse from each group, were used to ascertain the subcellular localization of the expressed AGT protein by confocal microscopy (data not shown). Intense AGT immunofluorescence was observed in the peroxisomes of AGXT-AAV2/8 treated hepatocytes, showing colocalization with peroxisomal protein PMP70, but not with the mitochondrial marker (mitotracker).

Serum transaminases (alanine aminotransferase ALT/GPT and aspartate aminotransferase AST/GOT) were not elevated (58.7±6.2 and 39±6.9 U/L, vs. 56.3±10.8 and 37.9±4.1 U/L, respectively, in non-treated mice), and no liver lesions were detected on H&E sections. This is in agreement with the lack of liver damage or toxicity reported by others using this type of rAAV vector at similar doses [Nakai H, Fuess S, Storm T A, Muramatsu S, Nara Y, Kay M A: Unrestricted hepatocyte transduction with adeno-associated virus serotype 8 vectors in mice. J. Virol. 2005 79(1):214-24.PMID: 15596817], [Gregorevic P, Blankinship M J, Allen J M, Crawford R W, Meuse L, Miller D G, Russell D W, Chamberlain J S: Systemic delivery of genes to striated muscles using adeno-associated viral vectors. Nat. Med. 2004 August; 10(8):828-34. Epub 2004 Jul. 25. PMID: 15273747]. In addition, we did not detect any significant titer of AGXT antibody, by western blot, in serum samples of AGXT-AAV2/8 treated mice.

We carefully inspected all the livers of our mice, including histological analysis, and found no evidence of oncogenesis after 50 day follow-up, in agreement with a large long-term study that found no evidence for tumorigenesis in AAV-treated mice [Bell et al. “No evidence for tumorigenesis of AAV vectors in a large-scale study in mice.” Mol. Ther. 12.2 (2005): 299-306].

Previous studies have shown that AAV5 is a better vector for the transduction of human liver. In a second set of experiments, we used several doses of AAV5 vectors, devoid of WPRE sequence, at either 1.5×10¹³, 5×10¹², or 5×10¹¹ viral particles/kg body weight, and AAV8 vectors, with WPRE, at 5×10¹² viral particles/kg, in both female and male Agxt1KO mice (n=5 animals per group). The goal of this set of experiments was to compare the effect of gene transfer with AAV5 with respect to the results achieved with the serotype capable of transducing the most hepatocytes in mice, namely AAV8. We also wanted to evaluate possible gender differences in the response. Agxt1KO mice were placed in metabolic cages, fed oxalate-free diet and water adlibitum, allowed to adjust for 3 days, and oxalate excretion was measured in 24 hr. urine.

Purified, high titer (4.5−10×10¹¹ viral particles/ml) preparations of rAAV2-AGXT-2/5 and -2/8 vectors, as well as rAAV2-eGFP-2/5 vectors, were administered to adult Agxt1KO mice, 12-16 weeks old, by single tail vein injections in 0.2-0.3 ml PBS-sucrose.

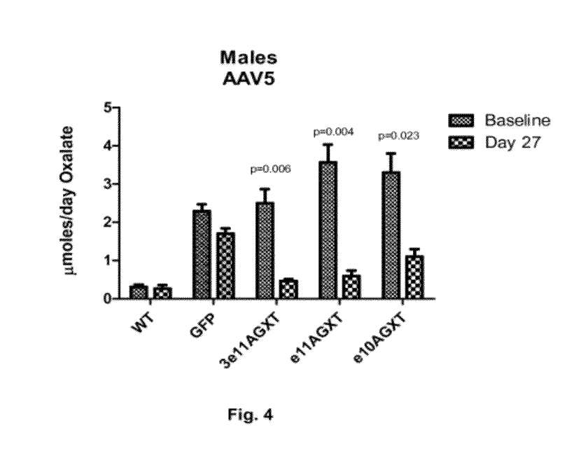

Urine oxalate excretion was followed during a total 35 day observation period, FIG. 4 shows the mean oxalate excretion for animals injected with either the human AGXT cDNA expressing vector or the GFP expressing control, at various doses of AAV5. All AGXT vector dose tested induced a significant reduction of oxalate excretion, compared with GFP controls. During the last week of the study, male mice were challenged with 0.5% ethylene glycol (EG) in drinking water, to follow the response to an overload of the glyoxylate cycle. The increases in oxalate excretion following the administration of the glyoxylate precursor were significantly lower in the groups receiving AGXT-AAV8 (0.44±0.49 μmmole/24 hrs) and the highest dose of AGXT-AAV5 (1.41±1.09 μmole/24 hrs) than in those injected with GFP-AAV5 (4.49±1.41 mole/24 hrs) (p=0.008 in both pairwise comparisons). On the other hand, the increases in oxalate excretion following the administration after the challenge of mice that received AGXT-AAV8 and the highest dose of AGXT-AAV5 were not significantly different from those observed in wild type mice (0.55±0.24 mole/24 hrs).

These results are consistent with the analysis of AGXT expression in the liver at the end of the study. Both AGT activity and western blot analysis showed higher levels of expression when using AAV8 vectors, compared with AAV5 vectors, both in males and females. Nevertheless, the levels of expression achieved with 10.5×10¹³ AAV5 particles/Kg body weight were consistently higher than those observed in wild type mice. Lower doses (5×10¹² and 5×10¹¹ AAV5 particles/kg) still resulted in significant AGT expression in male livers, while these doses resulted in relatively low signal in female livers.

Parallel results were obtained by immunohistochemical staining of frozen liver sections, with percentages of hepatocytes transduced around 90% for AAV8 and the highest dose of AAV5, 60% for 5×10¹² AAV5 particles/kg, and down to 10% (and less than 1% in females) for 5×10¹¹ AAV5 particles/kg (FIG. 3d).

Our experiments establish that gene transfer with vectors capable of transducing a large proportion of hepatocytes, such as rAAV2/8 and rAAV2/5, is a promising new therapy for primary hyperoxaluria type I. In this type of disease, where non-transduced hepatocytes would continue to produce oxalate, the therapeutic goal can only be achieved if enough hepatocytes are transduced so that the overall production of oxalate by the treated liver remains well below the capacity of the kidneys to excrete oxalate. The relatively large glomerular filtration rate (GFR) of the mouse, estimated at around 13 ml/min Kg body weight [Meneton P, Ichikawa I, Inagami T, Schnermann J. Renal physiology of the mouse. Am J Physiol Renal Physiol. 2000 March; 278(3):F339-51. Review. PMID: 10710537], might account for the lack of nephrocalcinosis in Agxt1KO mice, unless they are challenged with glyoxylate precursors. The administration of ethylene glycol, at doses that do not produce renal injury in the mouse, increases the oxalate excretion to levels leading to nephrocalcinosis in Agxt1KO mice, but not in wild type controls [Salido E C, Li X M, Lu Y, Wang X, Santana A, Roy-Chowdhury N, Tones A, Shapiro L J, Roy-Chowdhury J. Alanine-glyoxylate aminotransferase-deficient mice, a model for primary hyperoxaluria that responds to adenoviral gene transfer. Proc Natl Acad Sci USA. 2006; 103(48):18249-54]. Both rAAV2/8 and rAAV2/5, at doses above 5×10¹¹ viral particles/kg body weight are able to blunt the increase observed in mice drinking 0.5% ethylene glycol to levels similar to those observed in wild type mice. Similar results (not shown) were obtained with mice fed 0.5% glycolate in the diet.

Although AAV8 and high doses of AAV5 seem to correct the oxalate excretion similarly in males and females, substantially worse corrections were achieved in female mice injected with the lower doses of AAV5 constructs. Gender differences in expression of AAV vectors have been reported previously [Davidoff A M, Ng C Y, Zhou J, Spence Y, Nathwani A C. Sex significantly influences transduction of murine liver by recombinant adeno-associated viral vectors through an androgen-dependent pathway Blood. 2003 Jul. 15; 102(2):480-8; Pañeda A, Vanrell L, Mauleon I, Crettaz J S, Berraondo P, Timmerman E J, Beattie S G. Twisk J, van Deventer S, Prieto J, Fontanellas A, Rodriguez-Pena M S and Gonzalez-Aseguinolaza G. Effect of AAV serotype and genome structure over liver transduction and biodistribution in mice of both genders. Hum Gene Ther 2009, 20(8):908-17], but little is known about the mechanisms involved.

In summary, we showed that a single tail vein administration of rAAV2-AGXT-2/5 or 2/8 resulted in sustained correction of the PH1 mouse phenotype, without evidence of liver damage or toxicity.

This AAV-based gene transfer approach resulted in liver expression of the human AGXT cDNA sufficiently robust as to overcome the potential problem posed by the continued oxalate production of non-transduced hepatocytes. We also demonstrated that the product of the human AGXT gene is correctly targeted to the peroxisome in the mouse hepatocyte, and it complements the deficit of the mouse Agxt1KO model.

1-17. (canceled)

18. A method of treating an condition responsive to alanine-glyoxylate aminotransferase (AGXT), comprising administering to subject an effective amount of an AGXT I340M therapeutic.

19. The method according to claim 18, wherein the AGXT-responsive condition is primary hyperoxaluria type I.

20. The method according to claim 18, wherein the AGXT I340M therapeutic is selected from the group consisting of:
(a) an AGXT I340M protein comprising an amino acid sequence having at least 90% sequence identity to SEQ ID NO: 2 when optimally aligned over their entire length using the GAP program, and wherein the AGXT I340M protein comprises a methionine at a position corresponding to position 340 in SEQ ID NO: 2;
(b) a nucleic acid molecule comprising an nucleotide sequence encoding an AGXT I340M protein as defined in (a); and,
(c) a virion of a viral gene therapy vector comprising a nucleic acid molecule as defined in (b).

21. The method according to claim 20, wherein the AGXT I340M protein comprises an amino acid substitution selected from the group consisting of:
(i) a leucine at a position corresponding to position 11 in SEQ ID NO: 2;
(ii) an arginine at a position corresponding to position 41 in SEQ ID NO: 2;
(iii) an isoleucine at a position corresponding to position 152 in SEQ ID NO: 2;
(iv) an arginine at a position corresponding to position 170 in SEQ ID NO: 2;
(v) a threonine at a position corresponding to position 244 in SEQ ID NO: 2;
(vi) a threonine at a position corresponding to position 294 in SEQ ID NO: 2;
(vii) an isoleucine at a position corresponding to position 326 in SEQ ID NO: 2
(viii) combinations of one or more of the substitutions amino acid (ii) to (vii); and,
(ix) a combination of the substitutions (i) and (vi).

22. The method according to claim 18, wherein the AGXT I340M protein is selected from the group consisting of:
(a) an AGXT I340M protein comprising the amino acid sequence of SEQ ID NO: 2; and,
(b) an AGXT I340M protein comprising the amino acid sequence of SEQ ID NO: 2 and having one or more amino acid substitutions selected from the group consisting of:
(i) a leucine at a position corresponding to position 11 in SEQ ID NO: 2;
(ii) an arginine at a position corresponding to position 41 in SEQ ID NO: 2;
(iii) an isoleucine at a position corresponding to position 152 in SEQ ID NO: 2;
(iv) an arginine at a position corresponding to position 170 in SEQ ID NO: 2;
(v) a threonine at a position corresponding to position 244 in SEQ ID NO: 2;
(vi) a threonine at a position corresponding to position 294 in SEQ ID NO: 2;
(vii) an isoleucine at a position corresponding to position 326 in SEQ ID NO: 2
(viii) combinations of one or more of the substitutions amino acid (ii) to (vii); and,
(ix) a combination of the substitutions (i) and (vi).

23. The method according to claim 18, wherein the AGXT I340M therapeutic is a nucleic acid molecule comprising a nucleotide sequence encoding an AGXT I340M protein selected from the group consisting of:
(a) an AGXT I340M protein comprising the amino acid sequence of SEQ ID NO: 2; and,
(b) an AGXT I340M protein comprising the amino acid sequence of SEQ ID NO: 2 and having one or more amino acid substitutions selected from the group consisting of:
(i) a leucine at a position corresponding to position 11 in SEQ ID NO: 2;
(ii) an arginine at a position corresponding to position 41 in SEQ ID NO: 2;
(iii) an isoleucine at a position corresponding to position 152 in SEQ ID NO: 2;
(iv) an arginine at a position corresponding to position 170 in SEQ ID NO: 2;
(v) a threonine at a position corresponding to position 244 in SEQ ID NO: 2;
(vi) a threonine at a position corresponding to position 294 in SEQ ID NO: 2;
(vii) an isoleucine at a position corresponding to position 326 in SEQ ID NO:
(viii) combinations of one or more of the substitutions amino acid (ii) to (vii); and,
(ix) a combination of the substitutions (i) and (vi).

24. A nucleic acid construct comprising a nucleotide sequence coding for a AGXT I340M protein comprising: an amino acid sequence having at least 90% sequence identity to SEQ ID NO: 2 when optimally aligned over their entire length using the GAP program, and wherein the AGXT I340M protein comprises a methionine at a position corresponding to position 340 in SEQ ID NO: 2,
wherein the nucleotide sequence is operably linked to a promoter for expression in human cells and, optionally, wherein the promoter is not a promoter from a human AGXT gene.

25. The nucleic acid construct according to claim 24, wherein the promoter is a liver-specific promoter.

26. The nucleic acid construct according to claim 25, wherein the liver-specific promoter is selected from the group consisting of an α1-anti-trypsin (AAT) promoter, a thyroid hormone-binding globulin promoter, an albumin promoter, a thyroxin-binding globulin (TBG) promoter, an Hepatic Control Region (HCR)-ApoCII hybrid promoter, an HCR-hAAT hybrid promoter, an AAT promoter combined with the mouse albumin gene enhancer (Ealb) element and an apolipoprotein E promoter.

27. The nucleic acid construct according to claim 24, wherein the promoter comprises the sequence of SEQ ID NO: 3.

28. The nucleic acid construct according to claim 24, wherein the construct is a viral gene therapy vector.

29. The nucleic acid construct according to claim 28, wherein the viral gene therapy vector comprises a parvoviral vector.

30. A parvoviral virion comprising a nucleic acid construct according to claim 24.

31. A pharmaceutical composition comprising:
(a) an AGXT I340M protein comprising an amino acid sequence having at least 90% sequence identity to SEQ ID NO: 2 when optimally aligned over their entire length using the GAP program, and wherein the AGXT I340M protein comprises a methionine at a position corresponding to position 340 in SEQ ID NO: 2;
(b) a nucleic acid molecule comprising an nucleotide sequence encoding an AGXT I340M protein of (a);
(c) a virion of a viral gene therapy vector comprising a nucleic acid molecule of (b);
(d) a nucleic acid construct comprising a nucleotide sequence coding for a AGXT I340M protein comprising: an amino acid sequence having at least 90% sequence identity to SEQ ID NO: 2 when optimally aligned over their entire length using the GAP program, and wherein the AGXT I340M protein comprises a methionine at a position corresponding to position 340 in SEQ ID NO: 2, wherein the nucleotide sequence is operably linked to a promoter for expression in human cells and, optionally, wherein the promoter is not a promoter from a human AGXT gene; or
(e) parvoviral virion comprising the nucleic acid construct of (d), and
a pharmaceutically acceptable carrier.

32. A method for treating a condition responsive to AGXT comprising administering to a subject in need thereof an effective amount of an AGXT I340M therapeutic selected from the group consisting of:
(a) an AGXT I340M protein comprising an amino acid sequence having at least 90% sequence identity to SEQ ID NO: 2 when optimally aligned over their entire length using the GAP program, and wherein the AGXT I340M protein comprises a methionine at a position corresponding to position 340 in SEQ ID NO: 2;
(b) a nucleic acid molecule comprising an nucleotide sequence encoding an AGXT I340M protein of (a);
(c) a virion of a viral gene therapy vector comprising a nucleic acid molecule of (b);
(d) a nucleic acid construct comprising a nucleotide sequence coding for a AGXT I340M protein comprising: an amino acid sequence having at least 90% sequence identity to SEQ ID NO: 2 when optimally aligned over their entire length using the GAP program, and wherein the AGXT I340M protein comprises a methionine at a position corresponding to position 340 in SEQ ID NO: 2, wherein the nucleotide sequence is operably linked to a promoter for expression in human cells and, optionally, wherein the promoter is not a promoter from a human AGXT gene; and
(e) parvoviral virion comprising the nucleic acid construct of (d).


SEQUENCE LISTING (8 sequences)

SEQ ID NO: 1 - DNA, 1179 bp, Homo sapiens
  misc_feature: human AGXT I340M allele (rs4426527)
atggcctctcacaagctgctggtgaccccccccaaggccctgctcaagcccctctccatccccaaccagctcctgctggggcctggtccttccaacctgcctcctcgcatcatggcagccggggggctgcagatgatcgggtccatgagcaaggatatgtaccagatcatggacgagatcaaggaaggcatccagtacgtgttccagaccaggaacccactcacactggtcatctccggctcgggacactgtgccctggaggccgccctggtcaatgtgctggagcctggggactccttcctggttggggccaatggcatttgggggcagcgagccgtggacatcggggagcgcataggagcccgagtgcacccgatgaccaaggaccccggaggccactacacactgcaggaggtggaggagggcctggcccagcacaagccagtgctgctgttcttaacccacggggagtcgtccaccggcgtgctgcagccccttgatggcttcggggaactctgccacaggtacaagtgcctgctcctggtggattcggtggcatccctgggcgggacccccctttacatggaccggcaaggcatcgacatcctgtactcgggctcccagaaggccctgaacgcccctccagggacctcgctcatctccttcagtgacaaggccaaaaagaagatgtactcccgcaagacgaagcccttctccttctacctggacatcaagtggctggccaacttctggggctgtgacgaccagcccaggatgtaccatcacacaatccccgtcatcagcctgtacagcctgagagagagcctggccctcattgcggaacagggcctggagaacagctggcgccagcaccgcgaggccgcggcgtatctgcatgggcgcctgcaggcactggggctgcagctcttcgtgaaggacccggcgctccggcttcccacagtcaccactgtggctgtacccgctggctatgactggagagacatcgtcagctacgtcatggaccacttcgacattgagatcatgggtggccttgggccctccacggggaaggtgctgcggatcggcctgctgggctgcaatgccacccgcgagaatgtggaccgcgtgacggaggccctgagggcggccctgcagcactgccccaagaagaagctgtga

SEQ ID NO: 2 - protein, 392 aa, Homo sapiens
MASHKLLVTPPKALLKPLSIPNQLLLGPGPSNLPPRIMAAGGLQMIGSMSKDMYQIMDEIKEGIQYVFQTRNPLTLVISGSGHCALEAALVNVLEPGDSFLVGANGIWGQRAVDIGERIGARVHPMTKDPGGHYTLQEVEEGLAQHKPVLLFLTHGESSTGVLQPLDGFGELCHRYKCLLLVDSVASLGGTPLYMDRQGIDILYSGSQKALNAPPGTSLISFSDKAKKKMYSRKTKPFSFYLDIKWLANFWGCDDQPRMYHHTIPVISLYSLRESLALIAEQGLENSWRQHREAAAYLHGRLQALGLQLFVKDPALRLPTVTTVAVPAGYDWRDIVSYVMDHFDIEIMGGLGPSTGKVLRIGLLGCNATRENVDRVTEALRAALQHCPKKKL

SEQ ID NO: 3 - DNA, 673 bp, Artificial
  Human AAT promoter combined with the mouse albumin gene enhancer (Ealb) element
tcgaggttcctagattacactacacattctgcaagcatagcacagagcaatgttctactttaattactttcattttcttgtatcctcacagcctagaaaataacctgcgttacagcatccactcagtatcccttgagcatgaggtgacactacttaacatagggacgagatggtactttgtgtctcctgctctgtcagcagggcacagtacttgctgataccagggaatgtttgttcttaaataccatcattccggacgtgtttgccttggccagttttccatgtacatgcagaaagaagtttggactgatcaatacagtcctctgcctttaaagcaataggaaaaggccaacttgtctacgtttagtatgtggctgtagatctgtacccgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcacca

SEQ ID NO: 4 - DNA, 96 bp, Homo sapiens
tccgtcgacccgggcggccgatcgctagcctttgaatgtaaccaatcctactaataaaccagttctgaaggtgttgtgtgtgcgcgtgtggagttg

SEQ ID NO: 5 - DNA, 66 bp, Artificial
  Synthetic polyA Insulator
aattcaataaagagctcttattttcattctcgaggtgtggttggttttcttgtgtgggggcggatc

SEQ ID NO: 6 - RNA, 10 nt, Artificial
  Kozak consensus sequence
gccrccaugg

SEQ ID NO: 7 - DNA, 145 bp, adeno-associated virus 2
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 8 - DNA, 146 bp, adeno-associated virus 2
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcccc